(19) United States
(12) Patent Application Publication
(10) Pub. No.: US 2012/0289775 A1
(43) Pub. Date: Nov. 15, 2012

(54) POWER SUPPLY SYSTEM AND MEDICAL CAPSULE DEVICE MOUNTED WITH THIS POWER SUPPLY SYSTEM

(75) Inventor: Katsuyuki MURATA, Suwa-gun (JP)

(73) Assignee: OLYMPUS CORPORATION, Tokyo (JP)

(21) Appl. No.: 13/559,790

(22) Filed: Jul. 27, 2012

Related U.S. Application Data
(63) Continuation of application No. PCT/JP2010/071732, filed on Dec. 3, 2010.

(30) Foreign Application Priority Data
Jan. 27, 2010 (JP) 2010-015895

(51) Int. Cl. A61B 1/00; A61B 1/04; H01M 2/08; B82Y 30/00

(52) U.S. Cl. 600/104; 429/185; 600/109; 977/742; 977/734

(57) ABSTRACT

Provided are a power generation system which does not limit a patient's range of motion, is harmless to a living body, and can obtain a sufficient electricity generating capacity in the stomach or the intestines and a disposable medical capsule device which does not have to be collected after use. A power supply system is mounted in the capsule device, comprises an electrode pair including at least two electrodes provide on an outer wall surface of a capsule main body, for example, an aluminum electrode and a catalyst-supporting carbon electrode, generates power when immersed in an electrolytic solution consisting of gastric juice or intestinal juice, and supplies the power to constituent portions in the capsule device.

### CROSS-REFERENCE TO RELATED APPLICATIONS

This application is a Continuation Application of PCT Application No. PCT/JP2010/071732, filed Dec. 3, 2010 and based upon and claiming the benefit of priority from prior Japanese Patent Application No. 2010-015895, filed Jan. 27, 2010, the entire contents of all of which are incorporated herein by reference.

### BACKGROUND OF THE INVENTION

1. Field of the Invention

The present invention relates to an ingestion-type medical capsule device which is mounted with a power supply system that uses digestive contents present in a living body, generates electrical power, and performs ambient information detection and a medical procedure when flowing down in the living body.

2. Description of the Related Art

In recent years, various kinds of medical capsule devices formed into a capsule shape are known. As this capsule device, a typical capsule endoscope has an imaging mechanism and a battery mounted in a capsule and drives the imaging mechanism by the battery to perform imaging. The number of images to be taken is limited based on a capacity of the built-in battery, and introduction of a driving mechanism is also limited. Further, since a generally adopted small battery cannot be discarded as simple unburnable garbage, the battery is taken out from the capsule endoscope after discharged to the outside of a body and collected, and the battery is segregated and discarded.

As a countermeasure, in place of the battery, there has been also suggested a configuration that a power generation system or a power feed system is incorporated in a capsule device, the capsule device is administered, light, electrical waves, or electromagnetic waves are then transmitted from the outside of a living body, electrical energy is generated in the capsule device, and driving is carried out. For example, Jpn. Pat. Appln. KOKAI Publication No. 2001-231187 suggests a system that irradiates a medical capsule device with infrared light from the outside of a body and feeds electrical power. Moreover, Jpn. Pat. Appln. KOKAI Publication No. 9-327447 suggests a configuration where power generator is mounted in a capsule device.

Additionally, Jpn. Pat. Appln. KOKAI Publication No. 2007-200739 suggests a technology that uses an intragastric solution as an electrolytic solution (an electrolyte) and generates electrical power.

### BRIEF SUMMARY OF THE INVENTION

According to an aspect of embodiments, there is provided a power supply system comprising: a watertight capsule which has acid resistivity; at least one electrode pair which is provided to be exposed on an outer wall surface of the capsule and comprises different members having a difference in standard electrode potential; and a power supply unit which is arranged in the capsule, connected to the electrode pair, and takes out and supplies generated power, wherein the power supply system is constituted as a voltaic battery using digestive contents as an electrolytic solution.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1A is a view showing an external configuration of a medical capsule device according to a first embodiment of the present invention;

FIG. 1B is a view showing a cross-sectional configuration of the capsule device in a direction A-A in FIG. A;

FIG. 2 is a view showing a conceptual configuration of a power supply system mounted in the capsule device;

FIG. 3 is a view showing a cross-sectional shape of an electrode according to a first modification of a first embodiment in the direction A-A in FIG. 1A;

FIG. 4 is a view showing a cross-sectional shape of an electrode according to a second modification of the first embodiment in the direction A-A in FIG. 1A;

FIG. 5A is a view showing an external configuration of a capsule device according to a third modification of the first embodiment;

FIG. 5B is a view showing a cross-sectional configuration of an electrode in a direction B-B in FIG. 5A;

FIG. 6 is a view showing a cross-sectional configuration of a power supply system in a capsule device according to a fourth modification of the first embodiment;

FIG. 7 is a view showing a configuration where a temperature sensor is provided to an electrode of a power supply system according to a fifth modification of the first embodiment;

FIG. 8 is a view showing an external configuration of a capsule device having a power supply system mounted therein according to a second embodiment;

FIG. 9 is a view showing a cross-sectional configuration of a capsule device having a power supply system mounted therein according to a third embodiment;

FIG. 10 is a view showing an internal configuration of a capsule device having a power supply system mounted therein according to a fourth embodiment;

FIG. 11 is a view showing an internal configuration of a capsule device having a power supply system mounted therein according to a fifth embodiment;

FIG. 12 is a view showing an internal configuration of a capsule device having a power supply system mounted therein according to a sixth embodiment;

FIG. 13 is a view showing characteristics of a generated current obtained by a power supply system according to a seventh embodiment with respect to an elapsed time;

FIG. 14 is a view showing characteristics of the generated voltage obtained by the power supply system according to the seventh embodiment with respect to the elapsed time;

FIG. 15 is a view showing a relationship between an electrode area and the generated current in the power supply system according to the seventh embodiment;

FIG. 16 is a view showing characteristics of the generated current with respect to the elapsed time in an electrode pair of an aluminum electrode and a Pt electrode as a comparative example;

FIG. 17 is a view showing characteristics of a generated voltage with respect to an elastic time in the electrode pair of the aluminum electrode and the Pt electrode as a comparative example;

FIG. 18 is a view showing characteristics of the generated current obtained by a power supply system according to an eighth embodiment with respect to the elapsed time;

FIG. 19 is a view showing characteristics of the generated current obtained by a power supply system according to a modification of the eighth embodiment with respect to the elapsed time;

FIG. 20 is a view showing characteristics of the generated current obtained by a power supply system according to a ninth embodiment with respect to the elapsed time;

FIG. 21 is a view showing characteristics of the generated current obtained by a power supply system according to a tenth embodiment with respect to the elapsed time;

FIG. 22 is a view showing characteristics of the generated current obtained by a power supply system according to an eleventh embodiment with respect to the elapsed time;

FIG. 23 is a view showing an internal configuration of a capsule device having a power supply system mounted therein according to a twelfth embodiment;

FIG. 24A is a view showing an example of a manufacturing process of a laminated electrode formed on a capsule main body in the power supply system according to the twelfth embodiment;

FIG. 24B is a view showing an example of the manufacturing process of the laminated electrode following FIG. 24A;

FIG. 24C is a view showing an example of the manufacturing process of the laminated electrode following FIG. 24B;

FIG. 24D is a view showing an example of the manufacturing process of the laminated electrode following FIG. 24C;

FIG. 25A is a view showing another example of the manufacturing process of the laminated electrode in the power supply system according to the twelfth embodiment;

FIG. 25B is a view showing another example of the manufacturing process of the laminated electrode following FIG. 25A;

FIG. 25C is a view showing another example of the manufacturing process of the laminated electrode following FIG. 25B;

FIG. 25D is a view showing another example of the manufacturing process of the laminated electrode following FIG. 25C;

FIG. 25E is a view showing another example of the manufacturing process of the laminated electrode following FIG. 25D;

FIG. 25F is a view showing another example of the manufacturing process of the laminated electrode following FIG. 25E;

FIG. 25G is a view showing another example of the manufacturing processing of the laminated electrode following FIG. 25F;

FIG. 26 is a view showing characteristics of an electricity generating capacity with respect to a time in a battery using an artificial gastric juice as an electrolytic solution according to the first embodiment; and

FIG. 27 is a view showing characteristics of an electricity generating capacity with respect to a time in a battery using an artificial gastric juice as an electrolytic solution according to the first embodiment.

### DETAILED DESCRIPTION OF THE INVENTION

Hereinafter, embodiments of the present invention will be described below in detail with reference to the drawings.

### First Embodiment

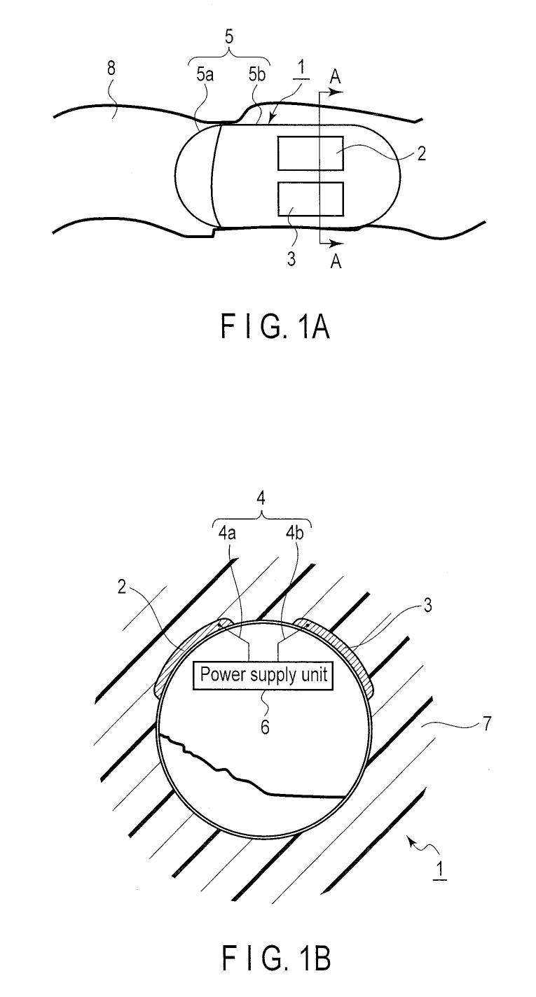

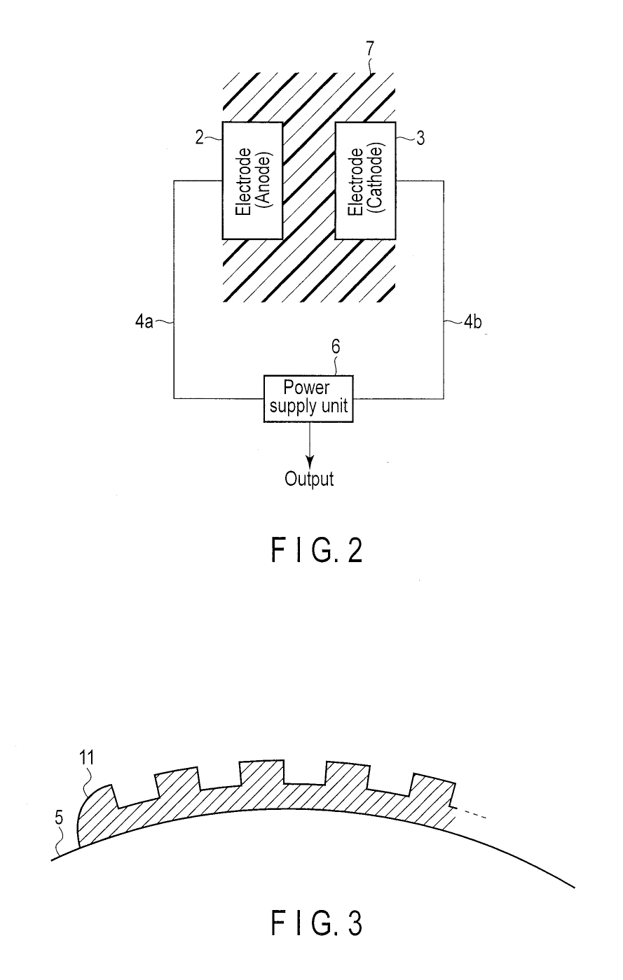

A concept of a power supply system according to the present invention will be first embodiment. FIG. 1A is a view showing an external configuration of an ingestion-type medical capsule device (which will be referred to as a capsule device hereinafter) administered in a living body according to a first embodiment of the present invention. FIG. 1B is a view showing a cross-sectional configuration of the capsule device in a direction A-A in FIG. 1A. Further, FIG. 2 is a view showing a conceptual configuration of a power supply system. FIG. 3 shows a cross-sectional shape of an electrode according to a first modification in the direction A-A in FIG. 1A.

In the following description, digestive contents suggest all materials included in a digestive organ which function as electrolytic substances or electrolytic solutions. Specifically, they are digestive secretions such as saliva or gastric juice secreted from a mouth, a stomach, an intestine, or the like, waste products from the digestive organ, or a mixture of materials taken from a mouth or a nose.

The capsule device 1 shown in FIG. 1A can have a shape that a patient can easily swallow at the time of administration into a living body 8. For example, like administration of a capsule containing medical agents, a bale-like shape having rounded front and rear parts is assumed. Of course, besides this shape, a spherical shape or a discoid shape can be easily applied in accordance with a device incorporated inside.

In this embodiment, a capsule main body 5 is divided into two parts, i.e., a front capsule 5a and a rear capsule 5b, and these capsules are water-tightly attached to configure the main body. At least the front capsule 5a is made of a transparent material that can form a window. For example, a resin material which is resistant to an acid such as a digestive secretion 7 is used for these capsule regions. Further, the entire capsule does not have to be formed of the same member, and it may be constituted of a combination of a transparent resin member (a window portion) and other members, for example, various different members such as a ceramic member.

A power supply system provided in the capsule device 1 will now be described.

The power supply system is constituted of at least two electrodes 2 and 3 which are provided on an outer wall surface of the capsule main body 5, entirely exposed and, and made of later-described different metals, and a power supply unit 6 which generates power of a predetermined constant voltage from electromotive force generated by these electrodes 2 and 3. Using these electrodes 2 and 3 and the surrounding digestive contents 7 as an electrolytic solution enables providing a known battery configuration called a voltaic battery. The power supply unit 6 uses electromotive force generated between the electrodes 2 and 3 utilizing this voltaic battery and supplies it as power to each non-illustrated drive unit of the capsule device 1.

In general, the electromotive force of the voltaic battery is determined based on a difference between standard electrode potentials of the electrodes made of two different types of metals. In the embodiment according to the present invention, as the electrode 2 serving as an anode, aluminum (including an aluminum alloy) as an amphoteric metal is selected. That is because an alkali metal or an alkali earth metal is preferable as an anode, but the cathode is administered into a body, an influence on a living body must be light, and the electrode should be one that can generate power even in the stomach having acidic properties or the duodenum having the basicity.

On the other hand, as the cathode 3, even if an electrode having a higher standard electrode potential than hydrogen is used, an actual electrode reaction is not a reaction of the electrode itself, and a reductive reaction of hydrogen ions in the electrode is dominant. Therefore, a material of the electrode 3 serving as the cathode does not have to be restricted in particular as long as the standard electrode potential is higher than the hydrogen. However, considering safety in a body, a noble metal such as platinum, gold, or silver or a carbon electrode is preferable. Although a carbon material used for the carbon electrode does not have to be restricted in particular if it has high conductive properties, as a specific example, it is possible to adopt conductive carbon black typified by acetylene black or ketjen black (a trade name: Lion), a composite of carbon black and amorphous carbon, graphite, carbon fiber, carbon nanotube, or a mixture of these materials.

It is to be noted that an area of the electrode 2 does not have to be necessarily equal to that of the electrode 3, and the electrode 2 may have an area larger than that of the electrode 3, for example. Further, in this embodiment, shapes of the electrodes 2 and 3 seen from above are rectangular shapes in FIG. 1A, but the shapes are not restricted as a matter of course, and any one of a circular shape, an elliptic shape, a track shape, a polygonal shape, and a triangular shape may be adopted, or these shapes may be combined to form each electrode. Besides these shapes, each of the electrodes 2 and 3 may be formed into a given pattern, for example, a comb-like shape so that comb teeth of these electrodes can mesh with each other to interpose spaces there between.

As a manufacturing method of these electrodes 2 and 3, a general deposition technology can be used. For example, as a dry deposition technology that is used in a semiconductor manufacturing technology, it is possible to easily apply evaporation deposition, sputtering deposition, ECR deposition, CVD deposition, or the like. Furthermore, as a wet film formation technology, plating film formation can be applied. Moreover, if the electrode is a carbon electrode, it can be formed by printing using, for example, a conductive carbon black ink. Additionally, the electrode may not be directly formed on the outer wall surface of the capsule main body 5, but the electrode may be additionally formed and then bonded to the outer wall surface of the capsule main body 5. As an adhesive, one used for medical purposes is preferable.

As described above, in the voltaic battery, polarization occurs, namely, electromotive force is reduced due to air bubbles of hydrogen generated in the cathode. To avoid this polarization, in the embodiment according to the present invention, when the configuration where the electrodes 2 and 3 are formed on the outer wall surface of the capsule device 1 as shown in FIG. 1A is adopted, the capsule device 1 flows down to prevent the solution on the electrode surface from staying, and the hydrogen air bubbles adhering to the electrode surface can be rapidly removed. It is to be noted that, as a conventional technique, a method of diffusing a depolarizer on the electrode 3 or a technique of using platinum that is hardly polarized is known. However, in a living body, using no depolarizer is desirable. Further, a polarization preventing effect of a platinum electrode is not sufficient either.

It is to be noted that, if the electrodes 2 and 3 are exposed on the wall surface of the medical capsule device 1, power can be generated without problem, and hence installing a shutter on each electrode when generating no power or covering each electrode with a thin film made of a digestible material for protection of the electrode has no problem in terms of functions as will be described later.

Furthermore, the power supply unit 6 is an integrated circuit having a general configuration, for example, a configuration that an amplification circuit and a constant voltage circuit (or a constant current circuit) are integrated and formed on one chip. It is to be noted that, to stabilize outputs, a capacitor (a charge storage element) that functions as a buffer may be arranged on an input side.

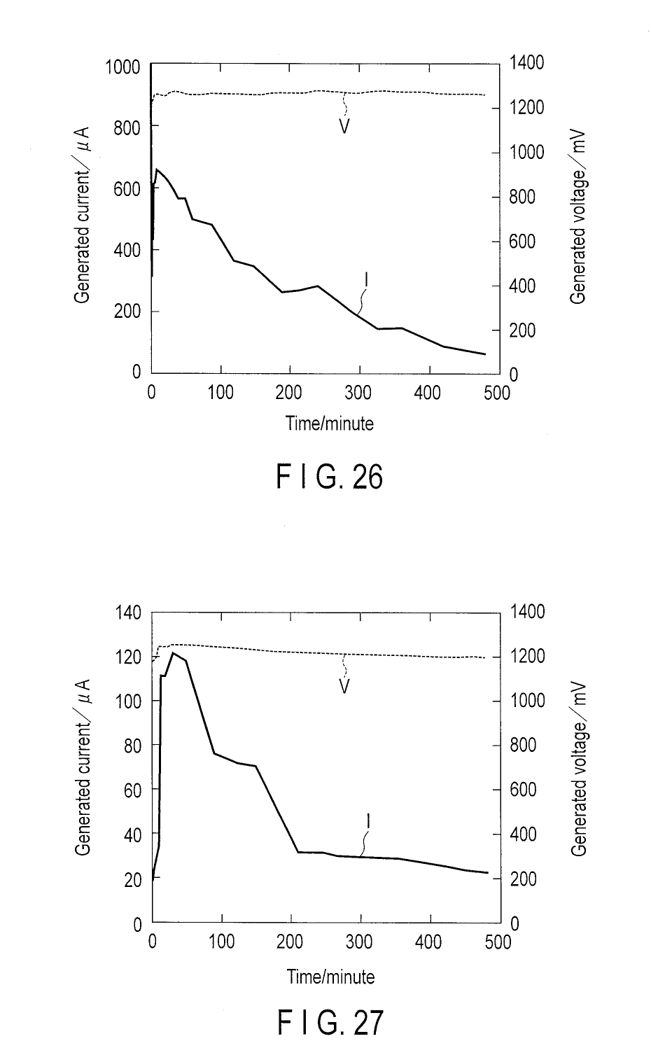

As shown in FIG. 26 and FIG. 27, an actual electricity generating capacity was verified using artificial gastric juice and artificial intestinal juice. 10 m of the artificial gastric juice and the artificial intestinal juice was put in a beaker made of glass, an aluminum electrode having an area of 1 cm² and a Pt electrode having an area of 0.02 cm² were immersed, and an electricity generating capacity (a generated current and a generated voltage) was checked.

Moreover, as a load of the battery, a resistor of 48 Ω was connected. In the artificial gastric juice, the maximum generated current was 0.7 mA, and an electricity generating capacity after elapse of 8 hours was 0.06 mA. The generated voltage was 1.25 V, and deterioration did not occur even after 8 hours.

In the artificial gastric juice, the maximum generated current was 0.12 mA, and the electricity generating capacity after elapse of 8 hours was 0.02 mA. The generated voltage was 1.2 V, and deterioration hardly occurred even after 8 hours.

A power supply system according to the first embodiment of the present invention will now be described.

The capsule device according to this embodiment is a power supply system that mainly operates in the stomach, and it has the same configuration as that depicted in FIG. 1B.

In the power supply system, on the outer wall surface of the capsule device 1 is formed an electrode pair including the electrode 2 made of aluminum functioning as an anode and the electrode 3 made of carbon functioning as an cathode, whereby a voltaic battery using intragastric contents as an electrolytic substance (an electrolytic solution) is configured. In the capsule device, the power supply unit 6 is arranged, and it is connected to the respective electrodes 2 and 3 through wiring lines 4a and 4b. These wiring lines 4a and 4b are welded (or soldered) and connected to parts of the electrodes 2 and 3 exposed on an inner wall of the capsule device. The power supply unit 6 is an integrated circuit having a general circuit configuration, for example, a configuration where an amplification circuit and a constant voltage circuit (or a constant current circuit) are integrated and formed on one chip. It is to be noted that, to stabilize outputs, a capacitor (a charge storage element) that functions as a buffer may be arranged on the input side.

The exposed portion on the electrode inner surface side can be provided by forming a hole in the capsule 5b from the inner side. For example, a hole (a bottomed hole) or a groove is previously formed in an electrode forming region of the capsule 5b from the outer side by cutting, and the electrode is formed to fill this hole by, for example, sputtering film formation. If a size of the capsule device 1 is, for example, 11 mm in diameter×26 mm long, each of the electrodes 2 and 3 is formed to have a size of approximately 10×10 mm. Then, when a thin bottom portion is chipped off from the inner side of the capsule 5b, the exposure can be easily effected.

As another method, a die that can be fitted on the inner side of this capsule 5b without gap is created. At least one through hole is formed in an electrode formation planned area at the time of forming the capsule 5b, and then the die is fitted on the inner side of the capsule. Subsequently, an electrode is formed to fill the hole from the outside, and the die is pulled out, whereby the electrode is exposed on the capsule inner side.

In the power supply system having such a configuration, since the stomach has gastric contents as one of digestive contents 7 and has acidic properties, an oxidative reaction of Al→Al³⁺+3e⁻ occurs in the electrode 2 by the voltaic battery using the gastric contents as an electrolytic solution, and a reductive reaction occurs in the electrode 3 by the same, whereby electromotive force is generated.

It is sufficient for these electrodes 2 and 3 to be exposed on the outer surface of the capsule main body 5 and immersed in the digestive contents, and these electrodes can be modified and changed in many ways. For example, when the electrodes 2 and 3 are buried in recesses or protrusions formed on the outer surface of the capsule main body 5, they are not restricted in particular as long as they are formed to be exposed on the surface. Further, the electrode 3 may be made of various kinds of noble metals such as platinum besides a carbon electrode.

Surface areas of these electrodes 2 and 3 can be designed based on the magnitude of current required by the capsule device 1. In case of consumption power in a capsule device which is an example currently in practical use, each electrode area of 0.25 cm² to 2 cm² is sufficient. Furthermore, if a larger current magnitude is required, each electrode surface area can be increased. In case of the capsule device which is in practical use, electrodes having a maximum area of approximately 6 cm² in total can be formed.

As a first modification of the first embodiment, a power supply system having electrodes having changed cross-sectional shapes will now be described. This modification has the same configuration as the power supply system in the first embodiment except the electrodes.

### First Modification of First Embodiment

A stripe electrode 11 in the first modification is formed into a stripe shape in which rectangular convex portions extending in a front-and-back direction of a capsule 5 are formed as a plurality of lines. As a forming method of this shape, the electrode may be formed with a large film thickness and then mechanically chipped off, or it is masked and then removed by anisotropic etching.

When the rectangular stripe electrode 11 is formed in this manner, not only a surface area which is in contact with digestive contents is increased, but also an area which is in contact with the digestive contents can be assured without closely attaching the stripe groove portions on the electrode surface to a gastric wall when coming into contact with the gastric wall (an intestinal wall). As a result, a reduction in electromotive force can be avoided. Further, when the capsule flows down, the digestive contents move toward the rear side along rectangular fins, and hence generated hydrogen bubbles can be efficiently removed.

Further, the stripe electrode 11 can also function as a heat sink. That is, the stripe electrode 11 also has a function of radiating from the electrode heat generated from respective drive portion of the capsule device. Therefore, an increase in surface area of the electrode is equal to an increase in area for heat radiation, and performance concerning cooling can be improved.

### Second Modification of First Embodiment

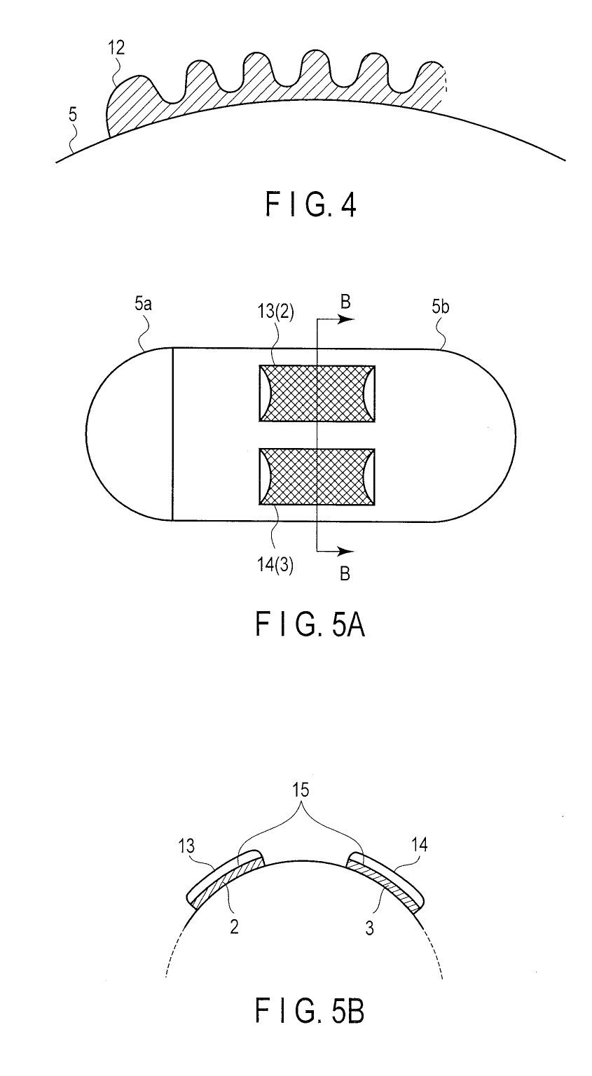

As a second modification of the first embodiment, a power supply system having electrodes having changed cross-sectional shapes will now be described. Except the electrodes, this modification has the same configuration as the power supply system according to the first embodiment. FIG. 4 shows a cross-sectional shape of an electrode according to the second modification of in the direction A-A in FIG. 1A.

With respect to the stripe electrode 11 having the rectangular cross section in the first modification, a stripe electrode 12 according to the second modification has a cross section formed into a curved surface. As a forming method, the electrode is formed with a large film thickness and then formed into a curved surface by isotropic etching such as wet etching. Of course, this electrode does not have to be necessarily formed into the curved surface, and a shape obtained by chipping off a side surface of the stripe into a tapered shape may be adopted.

This second modification can obtain the same functions and effects as those of the first modification.

### Third Modification of First Embodiment

As a third modification of the first embodiment, a power supply system with electrodes that has a function of holding digestive contents will now be described. This modification has a configuration equal to that of the power supply system according to the first embodiment except the electrodes. FIG. 5A is a view showing an external configuration of a capsule device according to a third modification. FIG. 5B is a view showing a cross-sectional configuration of electrodes in a direction B-B in FIG. 5A.

This third modification has a configuration that mesh covers 13 and 14 are provided to electrodes 2 and 3 while assuring separation spaces 15 having an arbitrary length. These mesh covers 13 and 14 have shapes covering the electrodes 2 and 3, openings of the separation space 15 are provided in a front-and-back direction (the shorter sides of each electrode depicted in FIG. 4), and each of the mesh covers 13 and 14 is connected on lateral surface sides.

These mesh covers 13 and 14 hold digestive contents 7 and function in such a manner that the electrodes 2 and 3 are constantly immersed in the digestive contents (or a digestive solution) 7 when staying in a living body. Further, the separation spaces 15 opened in the front-and-back direction are provided so that the new digestive contents 7 can constantly enter the separation spaces 15 when flowing down in the living body. When the digestive contents 7 flow through the separation spaces 15 from the front side toward the rear side, air bubbles generated on each electrode can be pushed toward the rear side.

Furthermore, a mesh size of each mesh cover 13 or 14 can be appropriately selected. Moreover, for example, in one mesh, a fine mesh is adopted on the front side so that the incoming digestive contents 7 can be readily held, and a coarse mesh is adopted on the rear side so that the air bubbles can flow out, namely, different mesh sizes may be combined. Moreover, an opening area on a front inlet side for the digestive contents 7 may be set larger than an opening area on an exit side for the same, the digestive contents 7 can be easily sucked and pushed out from the exit.

Additionally, in this modification, to hold the digestive contents 7, the mesh covers are adopted, but the present invention is not restricted thereto, and punch boards having many holes having an arbitrary diameter formed therein may be used.

It is to be noted that, for contribution to generation of electricity, the same material as a metal (conductive) member, for example, the electrodes can be used for the mesh covers 13 and 14. On the other hand, when just holding the digestive contents 7, the mesh covers 13 and 14 may be made of any other member, for example, a resin material.

As described above, since the mesh covers 13 and 14 are provided on the electrodes 2 and 3 to form the gaps, the electrodes 2 and 3 can be constantly immersed in the digestive contents when staying in the living body, thereby stably generating electricity. Further, when the mesh covers 13 and 14 are made of the same metal material as the electrodes 2 and 3, the magnitude of current to be generated can be increased without raising an occupied area for formation of the electrodes on the capsule main body 5.

### Fourth Modification of First Embodiment

As a fourth modification of the first embodiment, a power supply system having electrodes as anodes and cathodes will now be described. This modification has a configuration equal to that of the power supply system in the first embodiment except the electrodes.

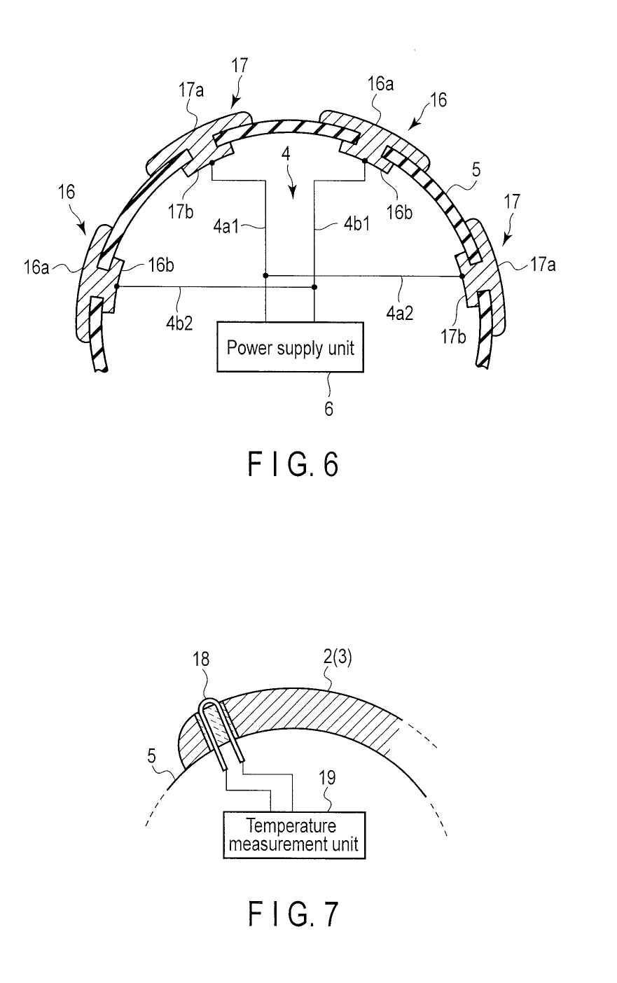

FIG. 6 is a view showing a cross-sectional configuration of the power supply system in a capsule device 1 according to the fourth modification.

In the fourth modification, electrodes (anodes) 16 and electrodes (cathodes) 17 are alternately formed to surround the outer periphery of a capsule main body 5. Each of the electrodes 16 and 17 is formed into a bobbin shape that each of outer electrodes 16a and 17a provided on an outer wall surface side of the capsule main body 5 and each of inner electrodes 16b and 17b provided on an inner wall surface side pierce a hole or a groove to be integrated. Of course, an outer shape of each of the electrodes 16 and 17 can be appropriately selected.

Since each of the inner electrodes 16a and 17b has a certain level of area, it can be easily connected to a power supply unit 6 through a wiring line 4 (4a1, 4a2, 4a3, 4a4). Further, heat generated by a constituent portion provided in the capsule main body 5 is received by the inner electrodes 16a and 17b, transferred to the outer electrodes 16b and 17b through the holes or grooves, and discharged from the outer electrodes 16b and 17b.

As described above, based on such an electrode configuration, the heat generated by driving of the constituent portion in the capsule main body 5 can be efficiently discharged. It is to be noted that the first to third modifications can be easily applied to these electrodes 16 and 17.

### Fifth Modification of First Embodiment

As a fifth modification of the first embodiment, a structural example in which a temperature sensor is provided to an electrode in a power supply system will now be described with reference to FIG. 7.

In the first to fourth modifications, the description has been given as to the configuration that the electrodes 2 and 3 have the heat radiating function. This modification has a configuration that a sensor which measures a temperature, for example, a thermocouple sensor 18 is provided to protrude or to be exposed from a surface of one electrode 2 (3) and a temperature measurement unit 19 is arranged in the capsule main body 5. In case of providing the thermocouple sensor 18 to the electrode 2 (3), it is desirable to provide a heat insulation material to surround the sensor and eliminate influence of heat (heat generated by driving of a constituent portion) from the electrode 2 (3).

According to this modification, it is possible to examine a temperature in a visceral tube which is a deep body temperature of a living body that cannot be measured in a regular state.

### Second Embodiment

A capsule device having a power supply system mounted therein according to a second embodiment will now be described.

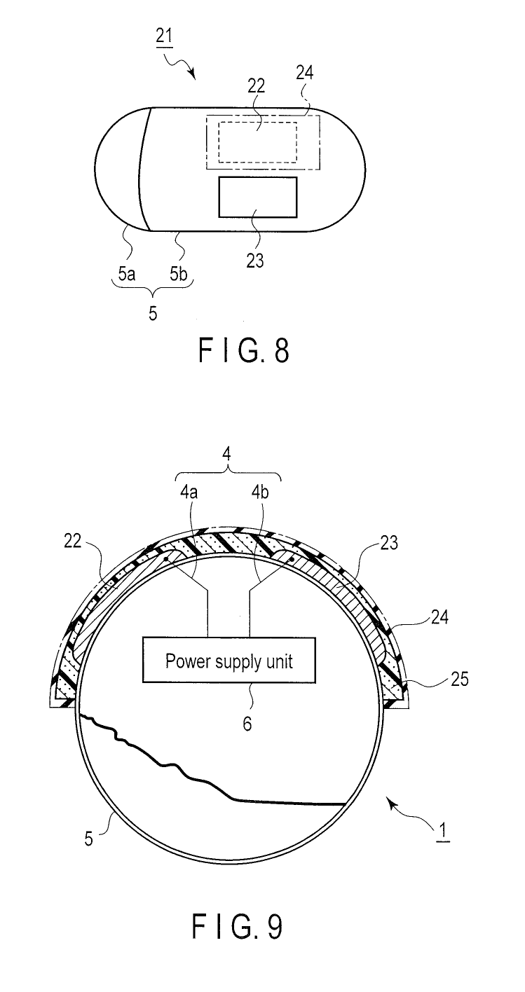

FIG. 8 shows an external structural example of a capsule device for enabling functioning mainly in the duodenum or the small intestine. Here, in regard to constituent portions in this embodiment, like reference numbers denote the same constituent portions as those in the first embodiment, and a description of these constituent portions will be omitted.

As the power supply system mounted in this capsule device 1, an electrode 22 made of aluminum that functions as an anode and an electrode 23 made of carbon that functions as an cathode are formed on a wall surface of a capsule main body 5, thereby constituting a voltaic battery using digestive contents as an electrolytic solution. The above-described power supply unit 6 is arranged in the capsule main body 5, and it is connected through wiring lines. Further, the electrode 21 is covered with a thin film (a gelatin film) 24 whose entire surface is made of a gelatin material. The capsule device 21 prevents gastric juice and others from adhering to the electrode 21 until the gelatin film 24 is dissolved when administered into a living body.

As a result, the capsule device 21 can reach the duodenum or the small intestine before the electrode 22 comes into contact with the gastric juice, and it can start power generation. A time required for the electrode 22 to be exposed after the gelatin film 24 is dissolved in this example can be adjusted by increasing or decreasing a thickness of the film based on information acquired from experiences or experiments. Therefore, after administration, power can be supplied to the capsule device 21 at a moment of reaching the duodenum or the small intestine.

In general, since trypsin which is a digestive enzyme effectively works in the duodenum, the duodenum is weakly basic with pH of approximately 8 to 9. The electrode 22 is made of aluminum in this embodiment because aluminum which is an amphoteric metal is oxidized even under such weakly basic conditions. Therefore, the electrodes 22 and 23 can function as a battery and supply power to the capsule device 21.

It is to be noted that the gelatin film 24 in this embodiment can be substituted by other materials. For example, besides gelatin, a thin film using starch, agar, or sugar may be adopted, and materials that can be dissolved or decomposed in digestive organs can be limitlessly used.

As described above, according to this embodiment, since the aluminum surface of the electrode 22 is coated, this electrode is not directly exposed to air (oxygen). Therefore, the electrode can be prevented from being oxidized when stored, and power generation that outputs a normal value can be performed immediately after dissolution of the thin film. Furthermore, since the electrode can be made of aluminum, a cost can be reduced as compared with a noble metal material. Moreover, in this embodiment, the electrodes or the mesh covers according to the first to third modifications can be likewise applied.

### Third Embodiment

A capsule device having a power supply system mounted therein according to a third embodiment will now be described.

FIG. 9 shows a cross-sectional configuration of a capsule device which mainly functions in the large intestine. Here, in regard to constituent portions in this embodiment, like reference numbers denote the same constituent portions as those in the first embodiment, and a description of these portions will be omitted.

A power supply unit 6 (not shown) of the power supply system in this capsule device 1 has the same configuration as that in the first embodiment.

In the capsule device 1 according to this embodiment, an electrode 22 made of aluminum functioning as an anode and an electrode 23 made of carbon functioning as an cathode are formed on an outer surface of a capsule main body 5, and a voltaic battery using digestive contents as an electrolytic solution is constituted. These electrodes are equal to those in the second embodiment. These electrodes 22 and 23 are covered with a porous thin film 25 which is configured to hold the solution, and they are further covered with a gelatin film 24.

Based on such as configuration, the gelatin film 24 prevents gastric juice or materials in the small intestine from adhering to the electrodes 22 and 23 without being dissolved until the capsule device 1 reaches the large intestine after administration. Additionally, when the capsule device 1 reaches the large intestine, the gelatin film 24 is dissolved, the digestive contents come into contact with the electrodes 22 and 23, and power generation is started. At this time, the porous thin film 25 holds the electrodes 22 and 23 in such a manner that these electrodes can be immersed in an electrolytic solution, and the electrodes 22 and 23 can be constantly immersed in the digestive contents even in a state that the amount of moisture of the electrolytic solution in the digestive contents is reduced in the large intestine, whereby power generation can be stably carried out.

Furthermore, using the gelatin film 24 enables obtaining the same effects as those in the second embodiment. Moreover, in this embodiment, the first to third modifications can be likewise applied.

### Fourth Embodiment

A capsule device having a power supply system mounted therein according to a fourth embodiment will now be described.

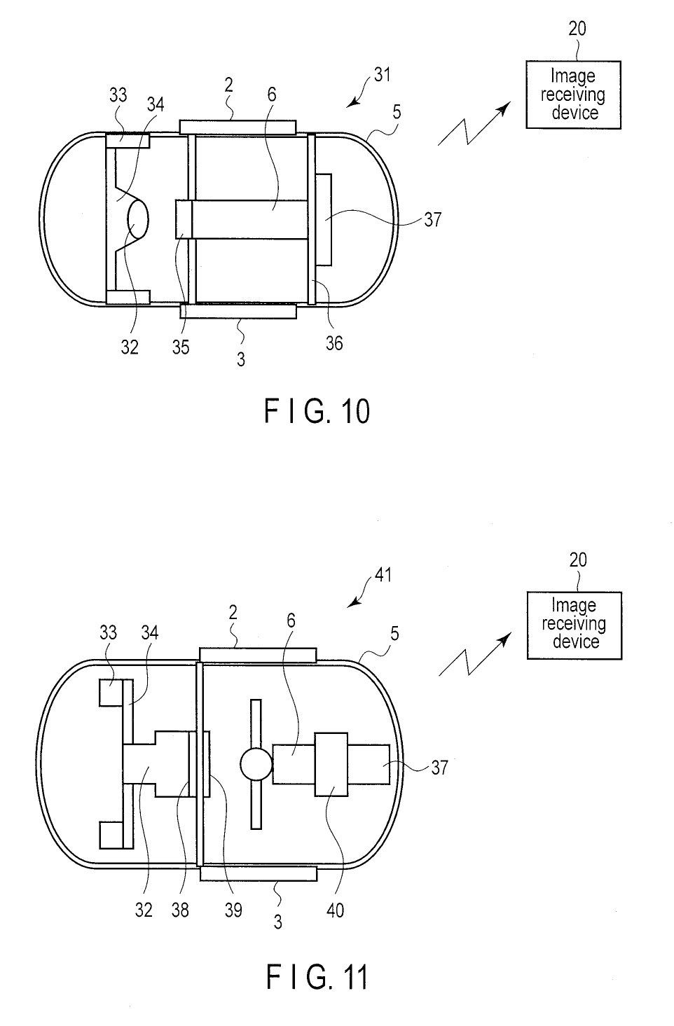

This embodiment is an example that the power supply system according to the present invention is applied to a capsule endoscope that operates in the small intestine. FIG. 10 shows an example of an internal configuration of a capsule endoscope 31. Here, in regard to constituent portions in this embodiment, like reference numbers denote the same constituent portions as those in the first embodiment, and a description of these constituent portions will be omitted.

A power supply system mounted in this capsule endoscope 31 is constituted of electrodes 2 and 3 and a power supply unit 6, and it is equal to the power supply system in each of the first to third embodiments and the first to fourth modifications. As to each of the electrodes 2 and 3, for example, a size of approximately 10 mm×10 mm is assumed.

The capsule endoscope 31 comprises a lens 32 configured to form an image of a shooting object (a subject) in a digestive organ after administered into a living body, light-emitting elements (for example, LEDs) 33 configured to illuminate a viewing field for shooting, a lens holder 34 that holds the lens 32 and the light-emitting elements 33, a CMOS sensor 35 that photoelectrically converts an optical image formed on the lens 32 and generates an image signal, an image processing unit, a wireless signal conversion unit (ASIC) 36, and an antenna 37.

The acquired image signal is generated as image data together with information concerning execution of various kinds of correction processing relating to noise elimination or a picture quality also concerning shooting, converted into a wireless communication information signal, and transmitted to an image receiving device 20 arranged outside the living body. In regard to transmission of an acquired image, the image may be constantly transmitted during a shooting period, or a memory may be mounted and the acquired image may be transmitted when temporarily stocked images reaches a given image amount, or the acquired image may be transmitted at predetermined time intervals.

The capsule endoscope 31 having this power supply system mounted therein is immersed in artificial gastric juice (which contains 0.2% of NaCl, 0.04% of pepsine, and 1M of HCl and is adjusted to PH 3.0) put in a constant-temperature tank having a temperature of 36° C. Power generated by the power supply system immersed in the artificial gastric juice is used, and respective constituent portions of the capsule endoscope 31 are driven, and imaging is carried out. Each acquired image is transmitted through the antenna 37 and displayed in a monitor (a viewer) of an external device, thereby confirming shooting and transmission of the image.

As described above, the power supply system according to the present invention is mounted in the capsule endoscope, and it can supply power in place of a conventional battery such as a silver oxide button battery.

### Fifth Embodiment

A capsule device having a power supply system mounted therein according to a fifth embodiment will now be described.

This embodiment is an example that the power supply system according to the present invention is applied to a capsule endoscope that operates in the small intestine. Here, in regard to constituent portions in this embodiment, like reference numbers denote portions that function in the same manner as the constituent portions in the fourth embodiment depicted in FIG. 10, and a description of these constituent portions will be omitted.

A power supply system mounted in this capsule device 41 is constituted of electrodes and a power supply unit, and it is equivalent to the power supply system according to each of the first to third embodiments and the first to fourth modifications.

The capsule endoscope 41 comprises a lens 32, light-emitting elements (LEDs) 33, a lens holder 34, a CCD 38 that photoelectrically converts an optical image formed on the lens 32 and generates an image signal, a digital signal processor (DSP) 39 that performs noise elimination and various kinds of correction processing with respect to the image signal, a wireless transmission module 40 that converts processed image data into a wireless signal, and an antenna 37.

An acquired image is generated as image data together with information concerning shooting in the DSP 39, converted into a wireless signal in the wireless transmission module 40, and transmitted to an image receiving device 20 arranged outside a living body through the antenna 37.

Like the fourth embodiment, this capsule endoscope 41 is immersed in the artificial gastric juice, power is generated from the power supply system, the capsule endoscope 41 is driven, and imaging is carried out. An acquired image is transmitted through the antenna 37 and displayed in a monitor of an external device (an image receiving device), thereby confirming shooting and transmission of the image.

As described above, the power supply system according to the present invention can be mounted in the capsule endoscope, and it can supply power in place of a conventional battery such as a silver oxide button battery. Further, as compared with a capsule endoscope having a conventional configuration, a space from which the battery has been removed is produced in the capsule endoscope according to this embodiment, and hence a reduction in size can be realized.

### Sixth Embodiment

A capsule device having a power supply system mounted therein according to a sixth embodiment will now be described.

The capsule device according to this embodiment is a capsule device having a drug delivery function unit that injects a drug solution to a target position or a sampling function unit that samples a gas and a liquid around an observation target and an endoscope function unit mounted in a capsule main body.

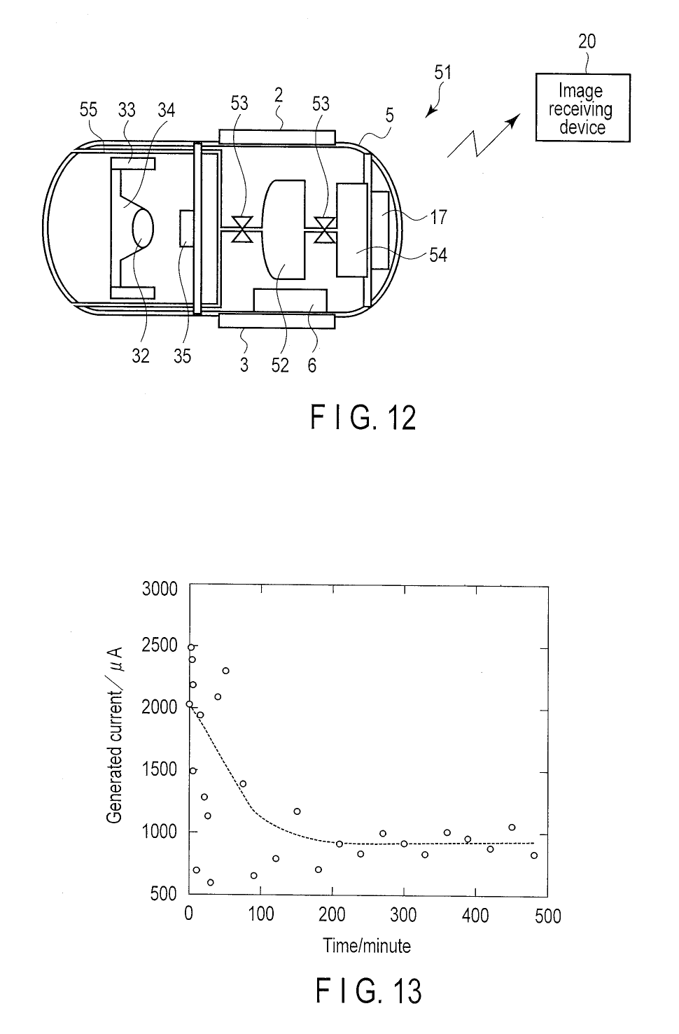

FIG. 12 shows an example of an internal configuration of a capsule device. Here, in regard to constituent portions in this embodiment, like reference numbers denote portions that function in the same manner as the constituent portions in the fourth embodiment depicted in FIG. 10, and a description of these constituent portions will be omitted.

A power supply system mounted in this capsule device 51 is constituted of electrodes 2 and 3 and a power supply unit 6, and it is equal to the power supply system according to each of the first to third embodiments and the first to fourth modifications.

The endoscope function unit in the capsule device 51 is mounted with the same configuration as that of the fourth embodiment. That is, the endoscope function unit comprises a lens 32, a light-emitting diode (an LED), a lens holder 34, a CMOS sensor 35, an ASIC 36, and an antenna 37.

Moreover, the drug delivery function unit is constituted of a drug solution cylinder 52 that accommodates an arbitrary drug solution, a magnetic valve 53, a compressed carbon dioxide gas tank 54, and a drug solution input nozzle 55. It is to be noted that the magnetic valve 53 is opened/closed in response to an instruction of a control unit (not shown) provided in the ASIC 36.

The capsule device 51 comprising the endoscope function unit and the drug delivery function unit observes an arbitrary image taken in a living body from the outside and determines an object region as a target. Subsequently, an instruction is given from the outside to open the magnetic valve 53 while confirming this image. When the magnetic valve 53 is opened, a compressed carbon dioxide gas pushes out a drug solution stored in the drug solution cylinder 52 to the drug solution input nozzle 55. This drug solution is sprayed and diffused from the drug solution input nozzle 55 toward the object region as the target displayed in a monitor of an image receiving device 2.

The drug solution adapted to this embodiment can be applied to not only a medical agent for cure but also various chemicals such as an enhancing agent for MRI or an anticancer agent.

Additionally, a capsule main body 5 comprises a pH sensor or a chemical sensor. Such a sensor may be used to perform not only a judgment using an image but also decision of the timing for inputting the drug solution based on a detection result from the pH sensor or the chemical sensor. As to the input timing, when a judgment value is preset with respect to a detection result of the pH sensor or the chemical sensor, automatic input can be also performed. For example, in case that the pH sensor is used, if a detected value of pH exceeds the judgment value when the capsule device 51 moves from the inside of the stomach where pH of digestive contents is low to the duodenum where pH of the same is high, the drug solution can be input.

Further, for the sampling function unit, a configuration of the drug delivery function unit can be used. That is, when the drug solution cylinder 52 is drained and the inside of the compressed carbon dioxide gas tank 54 is evacuated, the sampling function unit can sample a gas in a digestive tract and digestive contents.

The capsule device 51 comprising the endoscope function unit and the sampling function unit observes an arbitrary image shot in a living body from the outside and gives an instruction to open the magnetic valve 53 at a target position from the outside. When the magnetic valve 53 is opened, a gas and a liquid in a digestive organ are drawn into and stored in the drug solution cylinder 52 to cancel out a negative pressure formed due to the vacuum state in the compressed carbon dioxide gas tank 21. Subsequently, the endoscope function unit of the capsule device 51 is operated to obtain an image. Of course, the capsule device 51 may be discharged to the outside of the body without subsequent acquisition of an image.

As described above, according to the capsule device of the sixth embodiment, after the capsule device is administered into the body, when the magnetic valve 53 is opened when facing a desired object region as a target while observing an image acquired by the endoscope function unit, the drug solution can be sprayed to this object region. Moreover, in the same configuration, when the compressed carbon dioxide gas tank 21 is evacuated and the drug solution cylinder 52 is drained, opening the magnetic valve 53 at a desired position enables a gas and a liquid in a surrounding digestive organ to be drawn into the capsule main body, whereby the gas and the solution in the digestive organ can be sampled into the drug solution cylinder.

Additionally, when the power supply system according to this embodiment is adopted, a battery accommodation space in a capsule device having a conventional configuration can be assured, the drug delivery function unit can be mounted in this space and realized without changing a size of the capsule device.

It is to be noted that the description has been given as to the configuration that one of the sampling function unit and the drug delivery function unit is mounted in this embodiment, but a configuration having both these function units mounted therein may be adopted. Further, in this embodiment, although the drug delivery function unit is configured to spray the drug solution, it may be configured to belch a powdered medicine.

Furthermore, although FIG. 12 shows a state that the drug solution input nozzle 55 of the drug delivery function unit is opened, to prevent unnecessary digestive contents and the like from entering, a thin film that fractures by spraying a drug solution or vacuum suction may be provided at a nozzle opening, or an opening/closing lid may be provided.

The sampling function unit according to the sixth embodiment directly samples a gas in a digestive organ at a minimally-invasive shooting position. However, in a well-known technology, a gas in a digestive organ cannot be directly sampled at a minimally-invasive shooting position. Furthermore, in sampling of a gas from the anus, a composition of a digestive gas may be possibly changed when the digestive gas moves in a digestive organ. Therefore, the new sampling technique using the capsule device according to this embodiment enables taking out the most appropriate test object without damaging a living body at all by incision and the like, thereby providing a novel diagnostic method.

In the above-described power supply system according to the present invention, the structures in each embodiment and each modification are appropriately combined, elimination or modification of some of the constituent portions are included, and it stands to reason that this power supply system can be easily carried out. Moreover, the capsule device according to this embodiment is not restricted to being used alone, and it may be combined with any other examination device or medical device. For example, an X-ray imaging machine or the like may be also used, and a shooting position of an image acquired by the capsule device may be specified as required, for example. It is to be noted that the electrodes (the electrode pair) formed on the outer wall surface of the capsule device have electrode surfaces exposed to the outside in the first to sixth embodiments and the first to fourth modifications, the electrodes are not arranged to face each other and accommodated in a case like a configuration of Patent Literature 3, and an arrangement that the electrode surfaces face each other in the capsule main body and on the outer wall is not inclined.

### Seventh Embodiment

A power supply system provided in a capsule device 1 according to a seventh embodiment will now be described.

This power supply system has the same configuration as that of the capsule main body 5 in the first embodiment depicted in FIG. 1B, adopts a different electrode material, and uses as an electrolytic solution intestinal juice which is digestive contents present around electrodes, and generates power.

The power generation system is constituted of at least two electrodes 2 and 3 which are provided on an outer wall surface of a capsule main body 5, entirely exposed, and made of different metal materials, and a power supply unit 6 which generates power of a predetermined constant voltage from electromotive force produced by these electrodes 2 and 3.

As a power generation unit of this power generation system, when the electrodes 2 and 3 and digestive contents (intestinal juice) 7 present around the electrodes as an electrolytic solution are used, such a known battery configuration called a voltaic battery as depicted in FIG. 2 can be provided. The electromotive force generated between the electrodes 2 and 3 utilizing this voltaic battery is used, and the power supply unit 6 supplies this force as power to the respective constituent portions in the capsule device 1.

In general, the electromotive force of the voltaic battery is determined based on a difference in standard electrode potential between electrodes made of the two different types of metals. In this embodiment, for the electrode 2 serving as an anode, aluminum (including an aluminum alloy) as an amphoteric metal is selected. That is because an alkali metal or an alkali earth metal is preferable for the anode, but influence on a living body having the capsule device administered must be light, or the electrode must be able to generate power in the stomach having acidic properties and the duodenum having basicity.

On the other hand, in a regular voltaic battery, although a reductive reaction of hydrogen ions is dominant in the electrode 3 serving as an cathode, generation of hydrogen involved by this reaction can be not only a factor that reduces a generated current but also a generation source of a gas in the intestines since hydrogen adheres to the electrode. To avoid this inconvenience, in this embodiment, as a bio-voltaic battery, a power generation unit that uses the electrodes and substances in intestinal juice and generates power is suggested.

The hydrogen ions are used on the electrode 3 by a catalytic action, and enteric substances are reduced in terms of hydrogen adducts. In this reaction, a generated current higher than a reductive reaction of the hydrogen ions can be obtained. To efficiently perform this reaction, it is desirable for the electrode to have a wide surface area, the electrode must have safety with respect to the living body.

As the electrode meeting such demands, a carbon electrode is optimum. Although a carbon material used for the carbon electrode is not restricted in particular as long as it has high conductive properties, it is possible to adopt conductive carbon black typified by acetylene black or ketjen black (a trade name: Lion, a composite of carbon black and amorphous carbon, graphite, carbon fiber, carbon nanotube, carbon nanohorn, a mixture of these material, or a substance obtained by spraying such a material onto a conductive substrate such as carbon paper or carbon cloth as specific example.

To provide the carbon electrode of the electrode 3 with a catalytic action, a noble metal is supported on the carbon electrode. As the noble metal to be supported, gold, platinum, palladium, rhodium, or silver is preferable, in consideration of safety of a living body subjected to administration and characteristic of a reaction.

The capsule device according to this embodiment is mounted with a power supply system configured to mainly operate in the intestines.

In this power supply system, on the outer wall surface of the capsule device 1 is formed an electrode pair including the electrode 2 made of aluminum functioning as an anode and the electrode 3 made of carbon functioning as an cathode, whereby a voltaic battery using digestive contents as an electrolytic substance (an electrolytic solution) is configured.

In the capsule device, the power supply unit 6 is arranged, and it is connected to the respective electrodes 2 and 3 through wiring lines 4a and 4b. These wiring lines 4a and 4b are welded (or soldered) and connected to parts of the electrodes 2 and 3 exposed on an inner wall of the capsule device. The power supply unit 6 is an integrated circuit having a general circuit configuration, for example, a configuration where an amplification circuit and a constant voltage circuit (or a constant current circuit) are integrated and formed on one chip. It is to be noted that, to stabilize outputs, a capacitor (a charge storage element) that functions as a buffer may be arranged on the input side.

The exposed portion on the electrode inner surface side can be provided by forming a hole in the capsule 5b from the inner side. For example, a hole (a bottomed hole) or a groove is previously formed in an electrode forming region of the capsule 5b from the outer side by cutting, and the electrode is formed to fill this hole by, for example, sputtering film formation. If a size of the capsule device 1 is, for example, 11 mm in diameter×26 mm long, each of the electrodes 2 and 3 is formed to have a size of approximately 10×10 mm. Then, a thin bottom portion is chipped off from the inner side of the capsule 5b, the exposure can be easily effected.

As another method, a die that can be fitted on the inner side of this capsule 5b without gap is created. At least one through hole is formed in an electrode formation planned area at the time of forming the capsule 5b, and then the die is fitted on the inner side of the capsule. Subsequently, an electrode is formed to fill the hole from the outside, and the die is pulled out, whereby the electrode is exposed on the capsule inner side.

In the power supply system having such a configuration, a bio-voltaic battery using the digestive contents 7 as the electrolytic solution is configured, an oxidative reaction of Al→Al₃⁺+3e⁻ occurs in the electrode 2, and the digestive contents catalytically and reductively react like H⁺+A+e⁻→HA where A is a living substance contained in the intestinal juice in the electrode 3, whereby electromotive force is generated. As a substance that reductively reacts in the electrode 3, there is a substance voluminously contained in the intestinal juice like a taurocholic acid.

It is sufficient for these electrode 2 and 3 to be exposed on the outer wall surface of the capsule main body 5 and immersed in the digestive contents, and these electrodes can be modified and changed in many ways. For example, when the electrodes 2 and 3 are buried in recesses or protrusions formed on the outer surface of the capsule main body 5, they are not restricted in particular as long as they are formed to be exposed on the surface.

Furthermore, surface areas of these electrodes 2 and 3 can be designed based on the magnitude of current required by the capsule device 1. In case of consumption power in a capsule device which is an example currently in practical use, each electrode area of 0.25 cm² to 2 cm² is sufficient. Furthermore, if a larger current magnitude is required, each electrode surface area can be increased. In case of the capsule device which is in practical use, electrodes having a maximum area of approximately 6 cm² in total can be formed. Moreover, when irregularities are provided on the surface of the electrode 2 or porous aluminum such as formed aluminum is used for the electrode 2, a larger area can be provided.

A description will now be given as to an electricity generating capacity and power generation durability of the electrode 2 formed of the aluminum electrode and the electrode 3 formed of a Pt-supporting carbon electrode according to this embodiment.

First, to check the electricity generating capacity and the power generation durability, the electrodes 2 and 3 are immersed in an artificial intestinal juice, a load of, for example, 48 Ω is applied, and the electricity generating capacity is measured for 8 hours. It is to be noted that this load of 48 Ω is a numerical value conforming to a load when confirming the quality in a manufacturing process of a dry-cell battery. Additionally, the measurement time is set to 8 hours because it is an assumed time required for the capsule device to be discharged to the outside of a body from the stomach through the intestines after swallowed.

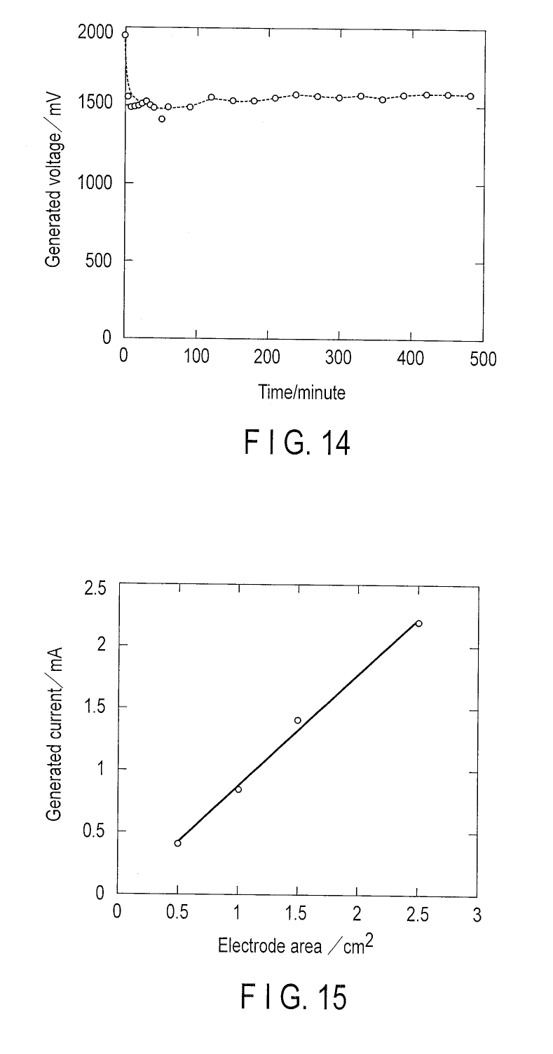

FIG. 13 is a view showing a generated current with respect to an elapsed time of each of the electrodes 2 and 3 according to this embodiment. FIG. 14 is a view showing characteristics of a generated voltage with respect to an elapsed time of each of the electrodes 2 and 3.

As shown in FIG. 13, although the generated current is precipitously reduced in 2 hours from the initial stage of power generation, power generation with a magnitude of current of 1 mA per cm² of an Al electrode area is thereafter stably carried out until 8 hours elapse. Further, as shown in FIG. 14, a generated voltage is not deteriorated when 8 hours elapse, and a voltage of 1.5 V is maintained.

Furthermore, FIG. 15 shows a relationship between an electrode area of each of the electrodes 2 and 3 and the generated current. Based on this result, the electrode area and the generated current have a proportional relationship, and increasing the electrode area leads to commensurately incrementing the generated current. Therefore, when the electrode area is increased in accordance with a necessary magnitude of current, the magnitude of generated current can be incremented.

### Comparative Example

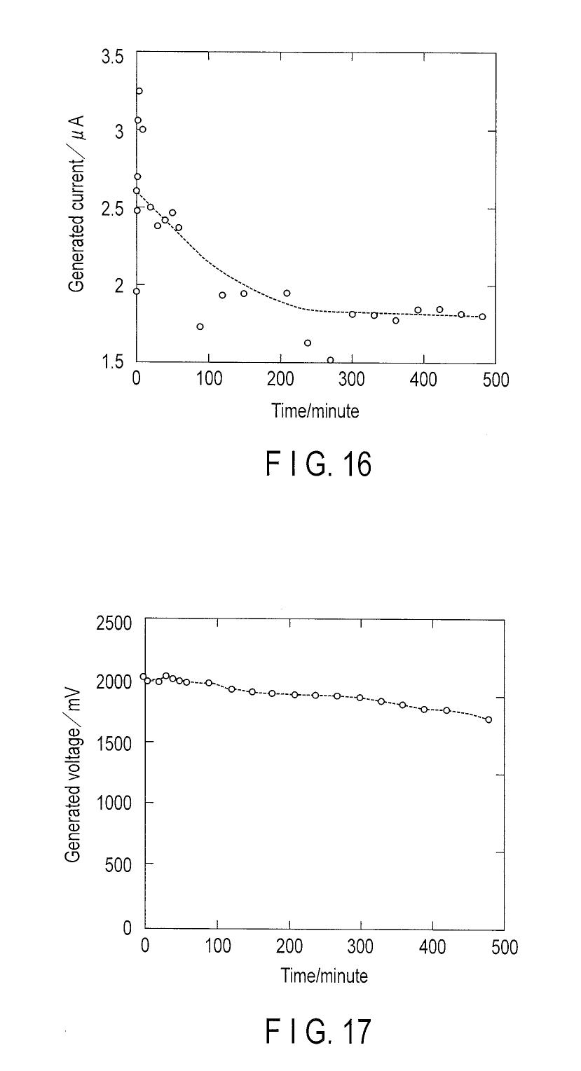

Each of FIG. 16 and FIG. 17 shows characteristics of a generated current and a generated voltage with respect to an elapsed time according to a comparative example for the electrodes 2 and 3 according to this embodiment. FIG. 16 shows a generated current with respect to an elapsed time in a comparative example where an aluminum electrode is applied to an electrode corresponding to the electrode 2 in this embodiment and a Pt electrode is applied to an electrode corresponding to the electrode 3. Additionally, FIG. 17 shows a generated voltage with respect to an elapsed time in this comparative example.

The aluminum electrode and the Pt electrode according to this comparative example are immersed in artificial intestinal juice, the same load of 48 Ω as that described above is applied, and an electricity generating capacity is measured until 8 hours elapse. The measured generated current is gradually reduced in approximately 3 hours from the initial stage of power generation, and power generation with a magnitude of current of 2 gA per cm² of an Al electrode area is thereafter stably carried out until 8 hours elapse. Degradation of the voltage is small even when 8 hours elapse, and a voltage of 1.5 V is maintained.

Therefore, both the generated voltage obtained by the electrode pair including the aluminum electrode 2 and the Pt-supporting carbon electrode according to this embodiment and the generated voltage obtained by the electrode pair including the aluminum electrode and the Pt electrode according to the comparative example fall within the range of 2 V to approximately 1.5 V. However, as the generated current according to this embodiment, a current value which is triple-digit larger than that of the generated current in the comparative example can be obtained. That is, since the electrode area is assured, this embodiment is in the practical stage.

### Eighth Embodiment

A power supply system according to the eighth embodiment will now be described.

This embodiment has a configuration including an electrode 2 formed of an aluminum electrode and an electrode 3 formed of an AuPt-supporting carbon electrode as electrodes in the power supply system. An electricity generating capacity and power generation durability in this electrode configuration will now be described.

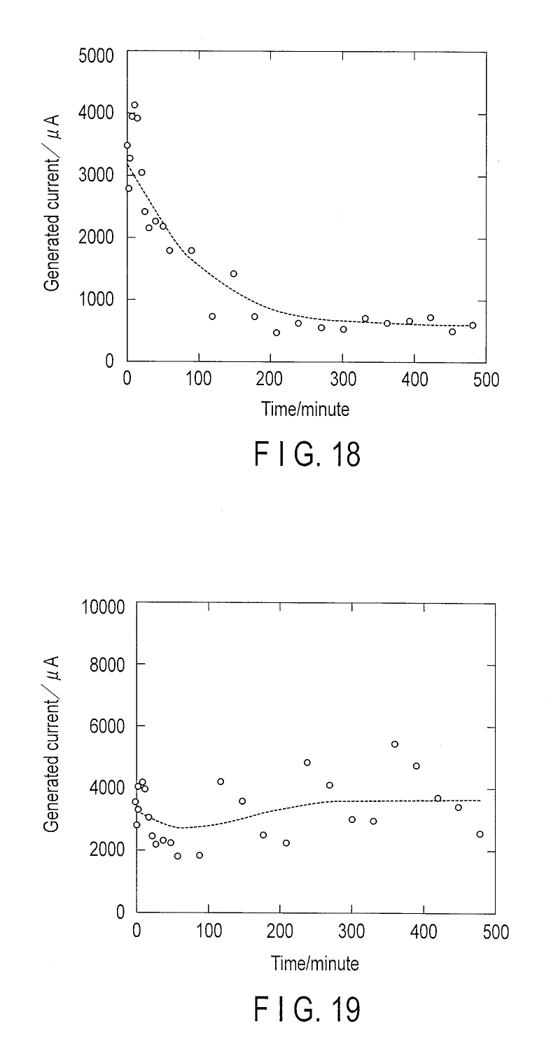

These electrodes 2 and 3 are immersed in artificial intestinal juice, a load of 48 Ω is applied, and an electricity generating capacity is measured in 8 hours from the initial stage of power generation. FIG. 18 is a view showing characteristics of a generated current with respect to an elapsed time obtained by the electrodes 2 and 3 according to this embodiment.

As shown in FIG. 18, the generated current is reduced from 4 mA to approximately 2 mA per cm² of an Al electrode area in approximately 3 hours from the initial stage of power generation, and power generation with a magnitude of current of 0.5 mA or above is thereafter stably carried out until 8 hours elapse. The maximum generated current is 4 mA. In regard to a generated voltage, like the first embodiment, deterioration such as a voltage drop is not observed until 8 hours elapse.

Since the electrode area is assured, the power supply system according to this embodiment is in the practical stage.

### Modification of Eighth Embodiment

Further, in this configuration, in regard to a reduction in generated current, a decrease in electrode area actually contributing the power generation is assured from any cause such as air bubbles produced in an cathode or oxidation of an Al electrode surface. It is to be noted that, in the measurement shown in FIGS. 13 to 15 and FIG. 18, electrodes 2 and 3 are put and immersed in artificial intestinal juice in a container. Therefore, this configuration is not administered into an actual living body, and it is not constantly in contact with new digestive contents 7 when flowing down. Furthermore, even if air bubbles or an oxide film is produced on the electrode, an operation of removing them is not carried out.

Therefore, in this modification, to obtain a fixed generated current, a predetermined electrode is added every predetermined time to add an electrode area, and power generation is carried out in such a manner that the electrode area contributing to the power generation is not reduced.

FIG. 19 is a view showing characteristics of a generated current when an aluminum electrode of 0.5 cm² is added every 2 hours from the start of power generation and the power generation is carried out in a situation where an aluminum electrode area in the initial stage of power generation is 0.5 cm².

In this example, the aluminum electrode area in the initial stage of power generation is 0.5 cm², and it is increased to 2 cm² after 8 hours. It can be understood that, when the area of the aluminum electrode is increased every given time, the generated current is restored to a current value on the initial stage of power generation immediately after the increase. Taking this situation as a whole, as the magnitude of current, an output of 3 mA is stably supplied as an average value from the initial stage of power generation to the elapse of 8 hours. Therefore, after the start of power generation, when the electrode is periodically added, the current value in the initial stage of power generation can be restored, and the generated current that is not lower than a given value can be continuously obtained.

Furthermore, as a technique of periodically increasing the electrode area, for example, the electrode surface is covered with, for example, thin films (gelatin films) with different thicknesses made of a gelatin material. The gelatin films are dissolved in the intestines with time differences, and the new electrode surface can be periodically exposed to the electrolytic solution in the digestive contents 7.

### Ninth Embodiment

A power supply system according to a ninth embodiment will now be described.

This embodiment has a configuration comprising an electrode 2 formed of an aluminum electrode and an electrode 3 formed of an Au-supporting carbon electrode as electrodes in the power supply system. An electricity generating capacity and power generation durability in this electrode configuration will now be described hereinafter.

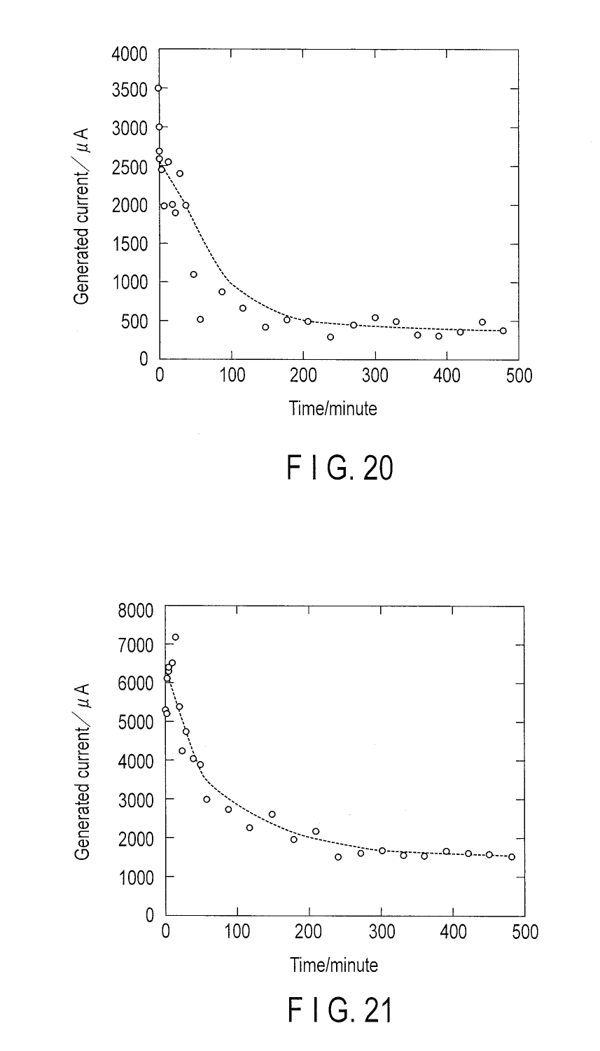

These electrodes 2 and 3 are immersed in an artificial intestinal solution, a load of 48 Ω is applied, and an electricity generating capacity in 8 hours from the initial stage of power generation is measured. FIG. 20 is a view showing characteristics of a generated current obtained by the electrodes 2 and 3 according to this embodiment with respect to an elapsed time.

As shown in FIG. 20, the magnitude of current is gradually reduced in approximately 3 hours from the initial stage of power generation, and then a magnitude of current of 0.5 mA is stably generated per cm² of an Al electrode area until 8 hours elapse. The maximum generated current is 3 mA. The voltage is not deteriorated even when 8 hours elapse. The power supply system according to this embodiment assures the electrode area, and hence it is in the practical stage.

### Tenth Embodiment

A power supply system according to a tenth embodiment will now be described.

This embodiment has a configuration comprising an electrode 2 formed of an aluminum electrode and an electrode 3 formed of a PdPt-supporting carbon electrode as electrodes in the power supply system. An electricity generating capacity and power generation durability in this electrode configuration will now be described hereinafter.

Although this PdPt-supporting carbon electrode has not been conventionally substantially used as an electrode of a fuel battery, using two types of noble metals for a bio-voltaic battery contributes to electromotive force or an increase in battery lifetime, and performance is improved. In particular, as combinations of the two types of noble metals, a combination of Pd and Pt is preferable.

These electrodes 2 and 3 are immersed in artificial intestinal juice, a load of 48 Ω is applied, and an electricity generating capacity in 8 hours from the initial stage of power generation is measured. FIG. 21 is a view showing characteristics of a generated current obtained by the electrodes 2 and 3 according to this embodiment with respect to an elapsed time.

As shown in FIG. 21, the magnitude of current is gradually reduced in approximately 3 hours from the initial stage of power generation, and then a magnitude of current of 1.5 mA is stably generated per cm² of an Al electrode area until 8 hours elapse. The maximum generated current is 7 mA. The voltage is not deteriorated even when 8 hours elapse. The power supply system according to this embodiment assures the electrode area, and hence it is in the practical stage.

### Eleventh Embodiment

A power supply system according to an eleventh embodiment will now be described.

This embodiment has a configuration comprising an electrode 2 formed of an aluminum electrode and an electrode 3 formed of a Pd-supporting carbon electrode as electrodes in the power supply system. An electricity generating capacity and power generation durability in this electrode configuration will now be described hereinafter.

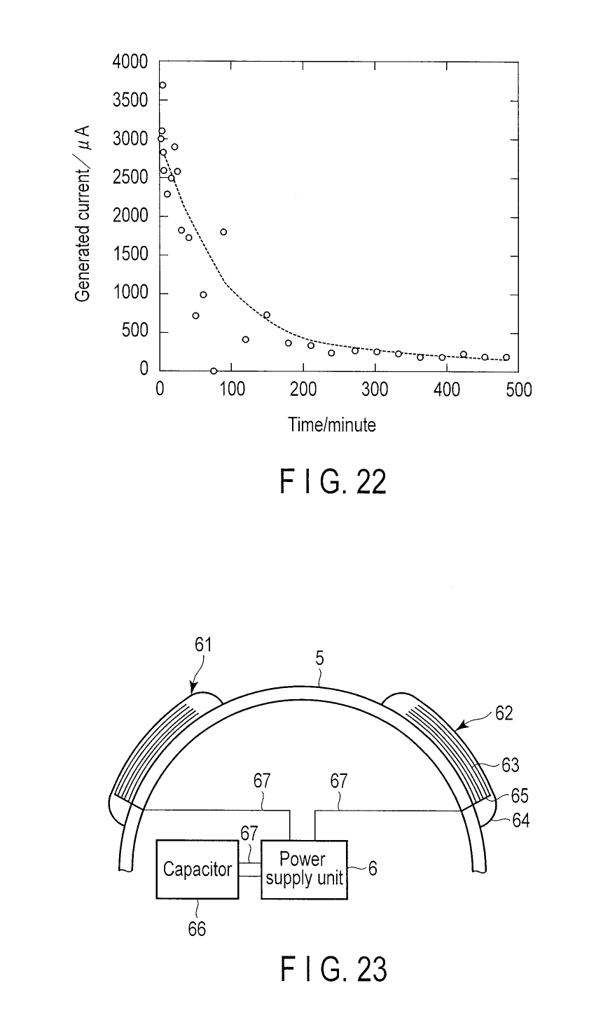

These electrodes 2 and 3 are immersed in an artificial intestinal solution, a load of 48 Ω is applied, and an electricity generating capacity in 8 hours from the initial stage of power generation is measured. FIG. 22 is a view showing characteristics of a generated current obtained by the electrodes 2 and 3 according to this embodiment with respect to an elapsed time.

As shown in FIG. 22, the magnitude of current is gradually reduced in approximately 3 hours from the initial stage of power generation, and then a magnitude of current of 0.3 mA is stably generated per cm² of an Al electrode area until 8 hours elapse. The maximum generated current is 3.5 mA. The voltage is not deteriorated even when 8 hours elapse.

### Twelfth Embodiment

In a configuration of each electrode in the power supply system according to each of the seventh to eleventh embodiments, in the initial stage of power generation, a generated current on the practical level can be obtained, but a value of this generated current tends to be reduced with time. As a cause of this tendency, it can be considered that the electrode surface which is in contact with the electrolytic solution of the digestive contents 7 changes and the changed electrode surface does not contribute to the power generation and that the electrolytic solution of the digestive contents 7 which is in contact with the electrode is stationary without flowing, the same electrolytic solution is in contact, and hence the appropriate power generation is not performed.

Since it is assumed that a capsule device having the power supply system mounted therein flows down in the intestines, it can be considered that an electrode pair (electrodes 2 and 3) formed on an outer surface of the capsule main body 5 constantly comes into contact with a new electrolytic solution in digestive contents 7. Therefore, the electrode pair does not become a problem since flowage with respect to the electrolytic solution in the digestive contents 7 is produced.

As shown in FIG. 23, the electrode pair according to this embodiment has multilayer electrodes 61 and 62 having the same configuration. As an electrode material forming the multilayer electrodes 61 and 62, a combination of the materials described in the seventh to eleventh embodiments is used.

The multilayer electrode 62 has a multilayer structure of electrode elements 63a to 63e and thin films (gelatin films) 64a to 64e made of an ultrathin gelatin material interposed between these electrode elements. Each of the electrode elements 63a to 63e is formed into a thin film or a mesh-like shape having small holes formed therein in such a manner that the interposed gelatin films 64a to 64e can be dissolved by an electrolytic solution in digestive contents 7. Therefore, to obtain a surface area of the electrode, the electrode elements 63a to 63e do not have to have a flat shape.

Moreover, the respective electrode elements 63a to 63e are electrically connected through a common wiring line 65. This common wiring line 65 is led into a capsule main body and connected to a power supply unit 6 through a wiring line 67, and it allows input of a generated current (voltage) to the power supply unit 6. Additionally, in this embodiment, there is concern that the electrode area exposed may be reduced by the remaining gelatin films and an electricity generating capacity may be decreased before the gelatin films 64a to 64e in the respective layers are completely dissolved, and hence providing a capacitor 66 functions to stably supply power during this reduction period.

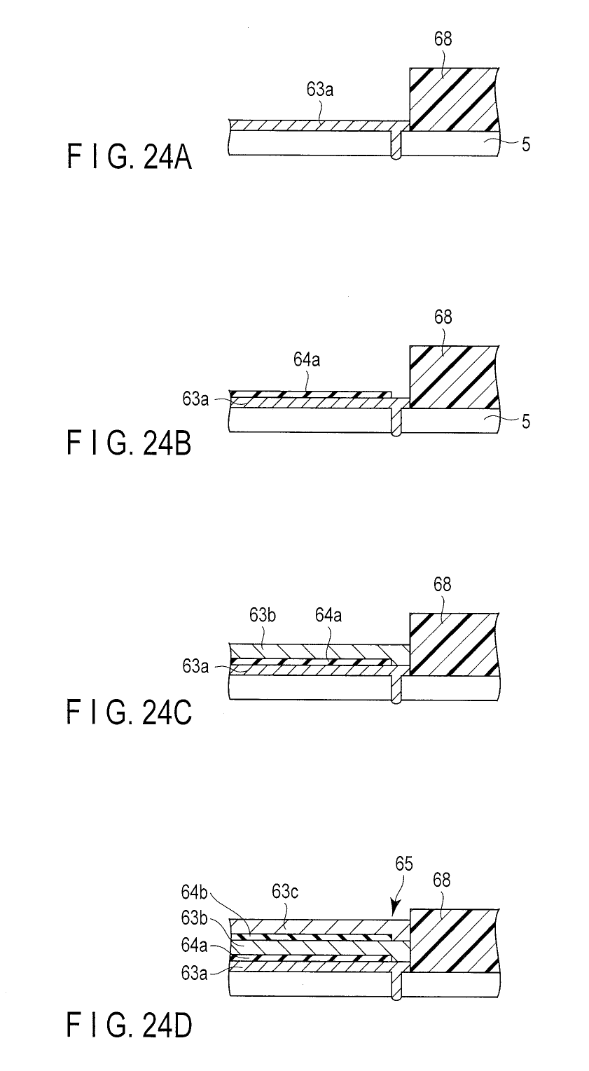

As shown in FIGS. 24A to 24D, the thus configured multilayer electrodes 61 and 62 are formed on an outer wall surface of the capsule main body 5. In FIG. 24A, the first electrode element 63a having a uniform film thickness is formed on the outer wall surface having a mask 68 formed thereon. The first electrode element 63a is formed to fill a hole provided in the capsule main body 5, and a portion formed in the hole serves as a leading line for a non-illustrated lower electrode.

Subsequently, as shown in FIG. 24B, a space serving as the common wiring line 65 is provided, and the first gelatin film 64a is formed. For example, this first gelatin film 64a can be printed using a print technology and then hardened to be formed. Thereafter, likewise, the multilayer structure is formed in a procedure of FIG. 24C and FIG. 24D.

As will be described later, the formed multilayer electrodes 61 and 62 may be additionally fabricated and attached to the capsule main body 5, or they may be directly formed on the outer wall surface. Further, a film thickness of the uppermost gelatin film is adjusted in such a manner that power generation starts when the device reaches the intestine. Based on this adjustment of the gelatin film, the multilayer electrodes 61 and 62 do not come into contact with gastric juice, and the capsule device 1 can reach the duodenum or the small intestine and start power generation.

When the capsule device having the multilayer electrodes 61 and 62 formed thereon flows down in the intestines, the uppermost electrode element 63e is exposed, power generation is started, and then the lower gelatin film 64d is dissolved after elapse of a give period. The electrode element 63e rises and is disconnected from the common wiring line 65, the next electrode element 63c is exposed, and power generation is started. Since the disconnected electrode element 63e is a small solid matter and thereafter discharged to the outside of a body, surrounding regions are not affected by the stay.

Then, the electrode exposed at that moment is disconnected every time the gelatin film 64 below the exposed electrode element 63 is dissolved, and the new electrode surface is sequentially exposed, and hence the same function as addition of a new electrode can be obtained as shown in FIG. 19, thereby avoiding a reduction in the electricity generating capacity and obtaining an output on the practical level close the electricity generating capacity in the initial level of power generation.

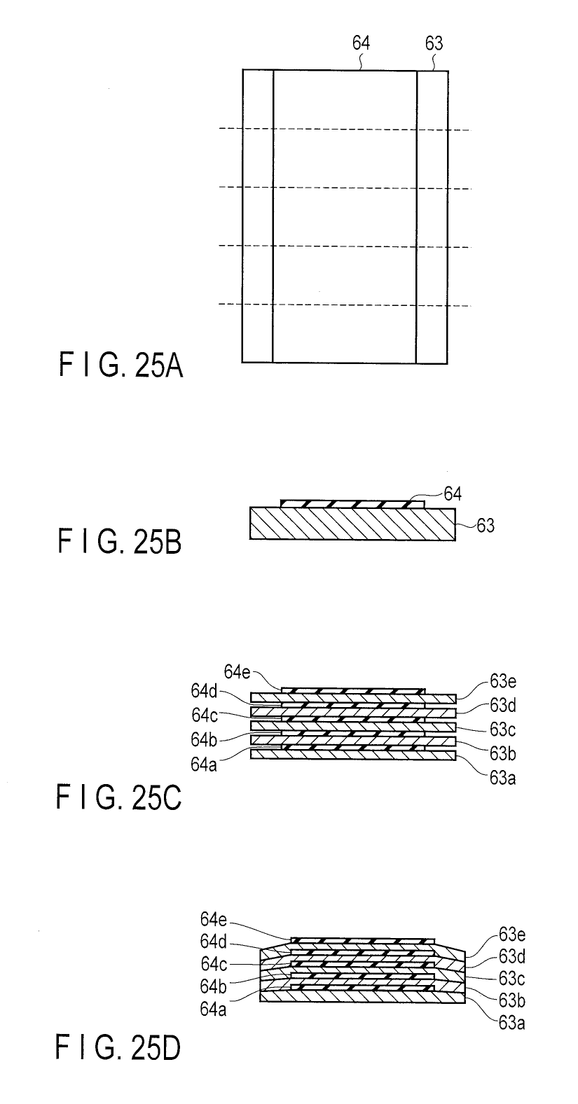

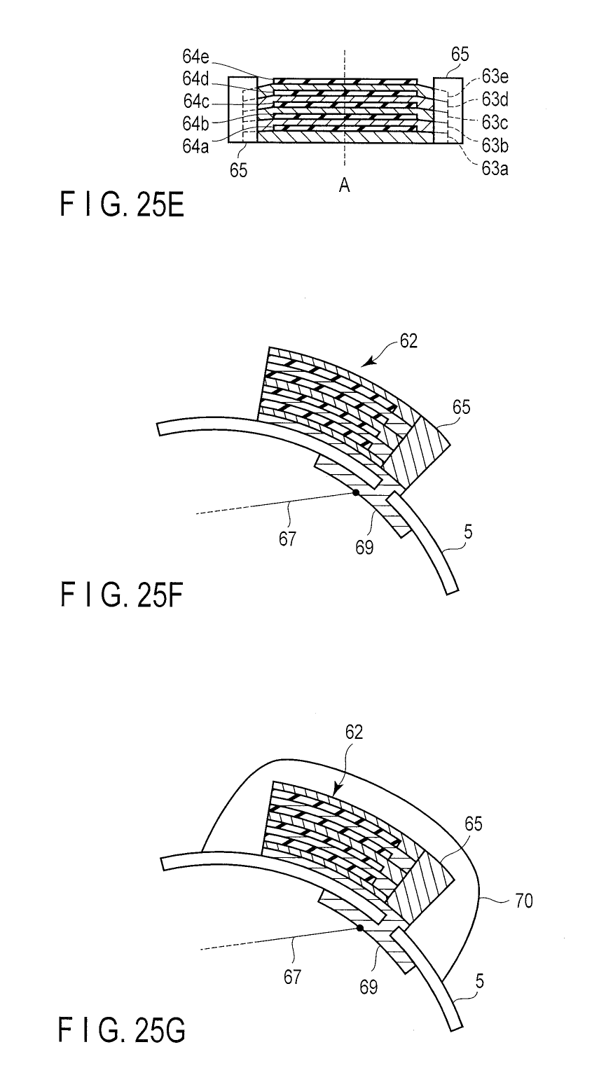

FIGS. 25A to 25G are views showing an example of a manufacturing process of the multilayer electrodes 61 and 62. In this example, one electrode film (for example, a Pt-supporting carbon film) is formed, cut, and laminated to form a multilayer electrode, and this electrode is bonded to a capsule main body. Here, a combination of the aluminum electrode 61 and the Pd-supporting carbon electrode 62 in the seventh embodiment is taken as an example.

First, as shown in FIG. 25A, based on a known manufacturing technology, a large Pd-supporting carbon thin film 63 corresponding to an area of electrodes is formed. Then, as shown in FIG. 25B, a gelatin film 64 having a predetermined thickness is formed while leaving both side ends of the Pd-supporting carbon thin film 63 to be exposed. Subsequently, this structure is cut at positions indicated by broken lines in FIG. 25A, electrode elements 63a to 63e are fabricated, and these electrode elements are laminated to form multiple stages as shown in FIG. 25C.

Then, as shown in FIG. 25D, in a state that both side ends of the electrode elements 63a to 63e are sandwiched from the upper and lower sides, surfaces of the gelatin films 64a to 64d are dissolved and then again hardened so that the electrode elements 63a to 63e are bonded to each other. Thereafter, as shown in FIG. 25E, a common electrode 65 is formed at each of both ends to include both the side ends of the electrode elements by using a technique such as plating, and the electrode is cut at the center indicated by a dotted line A.

Further, as shown in FIG. 25F, the electrode elements 63a to 63e are bonded to an underlying electrode 68 connected to the power supply unit 6 through a wiring line 67 to achieve electrical conduction. At this time, it is sufficient to electrically connect at least the common wiring line 65 to an underlying electrode 69.

As shown in FIG. 25G, after securing the electrode elements 63a to 63e, a gelatin film 70 is formed to cover the electrode elements 63a to 63e. As described above, a thickness of the gelatin film 70 is a thickness with which the power generation is started immediately after the device reaches the duodenum or the small intestine without coming into contact with the gastric juice, and it can be obtained on an experimental basis.

It is to be noted that, in the multilayer electrode 61 made of aluminum, the same manufacturing method may be used, or a known semiconductor film formation technology, for example, vapor deposition or CVD may be used. Moreover, the gelatin film according to this embodiment may be substituted by other materials. For example, besides gelatin, a thin film using starch, agar, or sugar may be adopted, and materials that are harmless (atoxic) to an examination target such as a living body and can be dissolved or decomposed in digestive organs can be limitlessly used.

It is to be noted that the multilayer electrode that the electrode element is exposed from the upper layer and then disconnected is adopted in this embodiment, but a layered electrode that the electrode element is not disconnected may be used. That is, a layered structure in which respective layers of electrode elements are connected to each other by supporting columns is formed, spaces between the layers are filled with gelatin, and the layered electrode in which the gelatin is exposed on side surfaces is formed. In the intestine, the gelatin may be dissolved and eliminated from the lateral sides of the layered electrode, and a surface area of the electrode may be increased with time. When this layered structure is adopted, a back side of the electrode element of each layer can function as an electrode surface contributing to power generation.

According to the present invention, it is possible to provide the disposable ingestion-type medical capsule device which has the built-in power generating battery that can obtain a sufficient electricity generating capacity in the stomach, the large intestine, and the small intestine of a living body where the medical capsule device flows down and which does not have to be collected after use.

The respective foregoing embodiments include the following invention:

(1) A power supply system comprising:
a watertight capsule which has acid resistivity;
at least one electrode pair which is provided to be exposed on a capsule outer wall surface and comprises two different members having a difference in standard electrode potential;
a holding member (13, 14) which covers the electrode pair to interpose an arbitrary distance between itself and the electrode pair and holds the digestive contents; and
a power supply unit which is arranged in the capsule, connected to the electrode pair, and takes out and outputs generated power,
wherein the power supply system is constituted as a voltaic battery using the digestive contents as an electrolytic solution.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A power supply system comprising:
a watertight capsule which has acid resistivity;
at least one electrode pair which is provided to be exposed on an outer wall surface of the capsule and comprises different members having a difference in standard electrode potential; and
a power supply unit which is arranged in the capsule, connected to the electrode pair, and takes out and supplies generated power,
wherein the power supply system is constituted as a voltaic battery using digestive contents as an electrolytic solution.

2. The system according to claim 1,
wherein, in the electrode pair,
a first electrode serving as an anode is made of aluminum, and
a second electrode serving as an cathode is made of either of a metal material containing platinum, gold, silver, and carbon or a carbon material.

3. The system according to claim 1,
wherein, in the electrode pair,
each electrode is formed into a stripe shape having convex lines in columns.

4. The system according to claim 1,
wherein, in the electrode pair,
when the electrodes are made of an amphoteric metal which is not resistant to the digestive contents, the electrodes have a thin film, which is made of a material having solubility with respect to the digestive contents and formed to have an arbitrary thickness, to cover the electrodes.

5. The system according to claim 1,
wherein, in the electrode pair,
the electrode pair is covered with a porous thin film which holds the digestive contents

6. The system according to claim 1, comprising:
an electrode pair in which:
a first electrode serving as an anode is an aluminum electrode; and
a second electrode serving as an cathode is a catalyst-supporting carbon electrode,
wherein the electrode pair is immersed in an electrolytic solution consisting of intestinal juice, and a power generation unit which generates power is thereby constituted.

7. A power supply system comprising:
a watertight capsule which has acid resistivity;
at least one electrode pair which is provided to be exposed on an outer wall surface of the capsule and comprises an aluminum electrode and a catalyst-supporting carbon electrode; and
a power supply unit which is arranged in the capsule, connected to the electrode pair, and takes out and supplies generated power,
wherein the power supply system is constituted as a voltaic battery using digestive contents as an electrolytic solution.

8. The system according to claim 6,
wherein the catalyst-supporting carbon electrode serving as an cathode in the electrode pair is a substance which supports noble metal fine particles on a conductive carbon material.

9. The system according to claim 8,
wherein a material used for a carbon electrode of the catalyst-supporting carbon electrode is one of conductive carbon black, a composite of carbon black and amorphous carbon, graphite, carbon fiber, carbon nanotube, carbon nanohorn, a mixture of these materials, and a material obtained by dispersing such materials onto a substrate which is made of carbon paper or carbon cloth and has conductive properties.

10. The system according to claim 8,
wherein the noble metal fine particles are one of gold, platinum, palladium, rhodium, and silver.

11. The system according to claim 6 or 7,
wherein the catalyst-supporting carbon electrode is formed of one of a Pt-supporting carbon electrode, an AuPt-supporting carbon electrode, an Au-supporting carbon electrode, a PdPt-supporting carbon electrode, and a Pd-supporting carbon electrode.

12. The system according to claim 6 or 7,
wherein each of the aluminum electrode and the catalyst-supporting carbon electrode forms a laminated electrode comprising:
electrode elements which are laminated;
interposed films which are interposed between the electrode elements and formed of a member which is dissolved by the digestive contents; and
a common wiring line which electrically connects one end of each of the electrode elements, and
when each interposed film is dissolved from the upper side, the exposed electrode element is disconnected from the common wiring line.

13. A medical capsule device comprising:
a watertight capsule which has acid resistivity;
a power supply system which comprises at least one electrode pair that is provided to be exposed on an outer wall surface of the capsule and comprises two different types of members having a difference in standard electrode potential, and takes out and supplies generated power;
a lens which forms an image of a shooting subject;
light-emitting elements configured to illuminate a viewing field for shooting;
an imaging sensor which photoelectrically converts an optical image formed by the lens and generates an image signal;
a processing unit which performs correction processing for the image signal generated by the optical sensor and converts the image signal into an information signal for wireless communication; and
an antenna which transmits the information signal from the processing unit to the outside.

14. The device according to claim 13,
wherein the medical capsule device further comprises a drug delivery function unit, the drug delivery function unit comprising:
a drug solution cylinder which stores an arbitrary drug solution;
a tank which compresses and accommodates a gas;
a nozzle from which the drug solution is belched toward the outside;
a first valve which is provided in a passage coupling the tank with the drug solution cylinder; and
a second valve which is provided in a passage coupling the drug solution cylinder with the nozzle,
the drug delivery function unit being configured to open the first and second valves at the timing conforming to an image acquired by the imaging sensor so that the gas belches out the drug solution stored in the drug solution cylinder from the nozzle.

15. The device according to claim 14,
wherein the medical capsule device further comprises:
the drug solution cylinder which is empty;
the tank in which a vacuum is formed; and
a sampling function unit which opens the first and second valves at the timing conforming to an image acquired by the imaging sensor, and draws into and stores in the cylinder one or both of a gas and a liquid which are present around the nozzle by using a negative pressure formed by the vacuum state in the tank.

16. A medical capsule device comprising:
a watertight capsule which has acid resistivity;
a power supply system which comprises an electrode pair that is provided to be exposed on an outer wall surface of the capsule and comprises an aluminum electrode and a catalyst-supporting carbon electrode, immerges the electrode pair in an electrolytic solution consisting of intestinal juice, and takes out and supplies generated power;
a lens which forms an image of a shooting subject;
light-emitting elements configured to illuminate a viewing field for shooting;
an imaging sensor which photoelectrically converts an optical image formed by the lens and generates an image signal;
a processing unit which performs correction processing for the image signal generated by the optical sensor and converts the image signal into an information signal for wireless communication; and
an antenna which transmits the information signal from the processing unit to the outside.

17. The device according to claim 16,
wherein each of the aluminum electrode and the catalyst-supporting carbon electrode which are provided to be exposed on the outer wall surface of the capsule forms a laminated electrode which comprises:
electrode elements laminated from the outer wall surface;
interposed films which are interposed between the electrode elements and formed of a member that is dissolved by the digestive contents; and
a common wiring line which electrically connects one end of each of the electrode elements and supplies generated power, and
when each interposed film is dissolved from the upper side, the exposed electrode element is disconnected from the common wiring line.
